(19) United States
(12) Patent Application Publication
(10) Pub. No.: US 2004/0265964 A1
(43) Pub. Date: Dec. 30, 2004

(54) Inducers of recombinant protein expression

(76) Inventors: Martin Allen, Seattle, WA (US); Pranhitha Reddy, Seattle, WA (US); James P. Boyce, Kirkland, WA (US); Jeffrey N. Fitzner, Seattle, WA (US)

Correspondence Address: IMMUNEX CORPORATION, LAW DEPARTMENT, 1201 AMGEN COURT WEST, SEATTLE, WA, 98119, US

(21) Appl. No.: 10/830,752

(22) Filed: Apr. 23, 2004

Related U.S. Application Data
(60) Provisional application No. 60/465,659, filed on Apr. 25, 2003.

(51) Int. Cl. C12Q 1/68; C07H 21/04; C07K 16/44; C12N 5/06

(52) U.S. Cl. 435/69.1; 435/320.1; 435/326; 435/328; 530/388.15; 536/23.53

(57) ABSTRACT

The invention provides methods of increasing the production of polypeptides, optionally recombinant polypeptides, from mammalian cells using an aromatic carboxylic acid, an acetamide, and/or a hydroxamic acid compound, and cultures containing the same.

### CROSS-REFERENCE TO RELATED APPLICATION

This application claims the benefit under 35 U.S.C. §119 of U.S. Provisional Application Ser. No. 60/465,659, filed Apr. 25, 2003.

### FIELD OF THE INVENTION

The invention is in the field of polypeptide production, particularly recombinant polypeptide production in cell culture.

### BACKGROUND

Polypeptides are useful in a variety of diagnostic, therapeutic, agricultural, nutritional, and research applications. Although polypeptides can be isolated from natural sources, the isolation of large quantities of a specific polypeptide from natural sources may be expensive. Also, the polypeptide may not be of uniform quality due to variation in the source material. Recombinant DNA technology allows more uniform and cost-effective large-scale production of specific polypeptides.

One goal of recombinant polypeptide production is the optimization of culture conditions so as to obtain the greatest possible productivity. Incremental increases in productivity can be economically significant. Some of the methods to increase productivity in cell culture include using enriched medium, monitoring osmolarity during production, decreasing temperatures during specific phases of a cell culture, and/or the addition of sodium butyrate (see, e.g., U.S. Pat. No. 5,705,364).

However, as more polypeptide-based drugs demonstrate clinical effectiveness and increased commercial quantities are needed, available culture facilities become limited. Accordingly, there remains a need in the art to continually improve yields of recombinant polypeptides from each cell culture run.

### SUMMARY

As shown by the experimental data reported herein, aromatic carboxylic acids, acetamides and/or hydroxamic acids are compounds that can dramatically induce the production of polypeptides, especially recombinant polypeptides, from mammalian cell lines. Moreover, these compounds can be used in combination, with each other and/or with other induction methods, to further increase polypeptide expression.

### BRIEF DESCRIPTION OF THE FIGURE

FIG. 1 is a graph of the Effect of Combinations of Compounds on Induction of Reporter Gene. Pools of cells that expressed a fluorescent marker protein, DsRed, under the control of an EASE/CMV promoter were cultured in 96 well plates at 35° C. for 6 days in the presence of the indicated amount of compound. The amount of fluorescence is plotted as a function of the concentration of compound. Compounds used for induction were hexanohydroxamic acid (HHA) (diamonds), Hexamethylenebisacetamide (HMBA) (squares) and both HHA+HMBA (triangles).

### DETAILED DESCRIPTION OF THE INVENTION

An “antibody” is a polypeptide or complex of polypeptides, each of which comprises at least one variable antibody immunoglobulin domain and at least one constant antibody immunoglobulin domain. Antibodies may be single chain antibodies, dimeric antibodies, or some higher order complex of polypeptides including, but not limited to, heterodimeric antibodies. A “human antibody” is an antibody encoded by nucleic acids that are ultimately human in origin. Such an antibody can be expressed in a non-human cell or organism. For example, DNA encoding a human antibody can be introduced into tissue culture cells and expressed in transformed cell lines. Alternatively, human antibodies can be expressed in transgenic animals such as, for example, the transgenic mice described in Mendez et al. ((1997), Nature Genetics 16(4): 146-56). Such transgenic mice are utilized in making the fully human antibodies in U.S. Pat. No. 6,235,883 B1. Human antibodies can also be expressed in hybridoma cells. A “humanized antibody” is a chimeric antibody comprising complementarity determining regions (CDR1, CDR2, and CDR3) from a non-human source and other regions that conform to sequences in human antibodies (and may be of human origin) as explained in, e.g., U.S. Pat. Nos. 5,558,864 and 5,693,761 and International Patent Application WO 92/11018.

A “constant antibody immunoglobulin domain” is an immunoglobulin domain that is identical to or substantially similar to a C_{L}, C_{H}1, C_{H}2, C_{H}3, or C_{H}4, domain of human or animal origin. See e.g. Hasemann and Capra, Immunoglobulins: Structure and Function, in William E. Paul, ed., Fundamental Immunology, Second Edition, 209, 210-218 (1989); Kabat et al., Sequences of Proteins of Immunological Interest, U.S. Dept. of Health and Human Services (1991).

An “F_{c} portion of an antibody” includes human or animal immunoglobulin domains C_{H}2 and C_{H}3 or immunoglobulin domains substantially similar to these. For discussion, see Hasemann and Capra, supra, at 212-213 and Kabat et al., supra.

Cells have been “genetically engineered” to express a specific polypeptide when recombinant nucleic acid sequences that allow expression of the polypeptide have been introduced into the cells using methods of “genetic engineering,” such as viral infection with a recombinant virus, transfection, transformation, or electroporation. See e.g. Kaufman et al. (1990), Meth. Enzymol. 185: 487-511; Current Protocols in Molecular Biology, Ausubel et al., eds. (Wiley & Sons, New York, 1988, and quarterly updates). Infection with an unaltered, naturally-occurring virus, such as, for example, hepatitis B virus, human immunodeficiency virus, adenovirus, etc., does not constitute genetic engineering as meant herein. The term “genetic engineering” refers to a recombinant DNA or RNA method used to create a host cell that expresses a gene at elevated levels or at lowered levels, or expresses a mutant form of the gene. In other words, the cell has been transfected, transformed or transduced with a recombinant polynucleotide molecule, and thereby altered so as to cause the cell to alter expression of a desired polypeptide. For the purposes of the invention, the antibodies produced by a hybridoma cell line resulting from a cell fusion are not “recombinant polypeptides.” Further, viral polypeptides produced by a cell as a result of viral infection are also not “recombinant polypeptides” as meant herein unless the viral nucleic acid has been altered by genetic engineering prior to infecting the cell. The methods of “genetic engineering” also encompass numerous methods including, but not limited to, amplifying nucleic acids using polymerase chain reaction, assembling recombinant DNA molecules by cloning them in Escherichia coli, restriction enzyme digestion of nucleic acids, ligation of nucleic acids, and transfer of bases to the ends of nucleic acids, among numerous other methods that are well-known in the art. See e.g. Sambrook et al., Molecular Cloning: A Laboratory Manual, 2^{nd} ed., vol. 1-3, Cold Spring Harbor Laboratory, 1989. Methods and vectors for genetically engineering cells and/or cell lines to express a polypeptide of interest are well known to those skilled in the art. Genetic engineering techniques include but are not limited to expression vectors, targeted homologous recombination and gene activation (see, for example, U.S. Pat. No. 5,272,071 to Chappel) and trans activation by engineered transcription factors (see e.g., Segal et al., 1999, Proc. Natl. Acad. Sci. USA 96(6):2758-63). Optionally, the polypeptides are expressed under the control of a heterologous control element such as, for example, a promoter that does not in nature direct the production of that polypeptide. For example, the promoter can be a strong viral promoter (e.g., CMV, SV40) that directs the expression of a mammalian polypeptide. The host cell may or may not normally produce the polypeptide. For example, the host cell can be a CHO cell that has been genetically engineered to produce a human polypeptide, meaning that nucleic acid encoding the human polypeptide has been introduced into the CHO cell. Alternatively, the host cell can be a human cell that has been genetically engineered to produce increased levels of a human polypeptide normally present only at very low levels (e.g., by replacing the endogenous promoter with a strong viral promoter).

“Growth phase” means a period during which cultured cells are rapidly dividing and increasing in number. During growth phase, cells are generally cultured in a medium and under conditions designed to maximize cell proliferation.

A “hybrid polar compound” is compound having two polar groups separated by an apolar carbon chain. This includes hexamethylene bisacetamide (HMBA) and the other molecules discussed in copending application Ser. No. 10/400,334 and in the following references: Richon et al. (1998), Proc. Natl. Acad. Sci. 95: 3003-07; Marks et al. (1994), Proc. Natl. Acad. Sci. 91: 10251-54; and U.S. Pat. Nos. 5,055,608 and 6,087,367.

The production of a polypeptide is “increased” by the addition of an inducing agent, such as aromatic carboxylic acid, acetamide, and hydroxamic acid compounds, if the amount the polypeptide produced in a culture containing the inducing agent is more than the amount of the polypeptide produced in an otherwise identical culture that does not contain the inducing agent. Similarly, the production of a polypeptide is “increased” by growth at a temperature other than 37° C. if the amount of polypeptide produced in a culture incubated at a temperature other than 37° C. is more than the amount of the polypeptide produced in an otherwise identical culture incubated at 37° C. Typically, the cell(s) exposed to the compound or inducing agent will be maintained in culture for at least about 2 days, and more typically about 5 to 10 days, and sometimes even longer, before the cells and medium are harvested and production of the polypeptide is assessed.

A “multimerization domain” is a domain within a polypeptide molecule that confers upon it a propensity to associate with other polypeptide molecules through covalent or non-covalent interactions.

A “naturally-occurring polypeptide” is a polypeptide that occurs in nature, that is, a polypeptide that can be produced by cells that have not been genetically engineered. Such a polypeptide may also be produced by cells genetically engineered to produce it.

“Polypeptide” means a chain of at least 6 amino acids linked by peptide bonds. Optionally, a polypeptide can comprise at least 10, 20, 30, 40, 50, 60, 70, 80, 90, 100, 150, 200, 250, or 300 amino acids linked by peptide bonds.

“Production medium” means a cell culture medium designed to be used to culture cells during a production phase.

“Production phase” means a period during which cells are producing maximal amounts of recombinant polypeptide. A production phase is characterized by less cell division than during a growth phase and by the use of medium and culture conditions designed to maximize polypeptide production.

A “recombinant fusion polypeptide” is a fusion of all or part of at least two polypeptides, which is made using the methods of genetic engineering.

A “recombinant polypeptide” is a polypeptide resulting from the process of genetic engineering. For the purposes of the invention, the antibodies produced by a hybridoma cell line resulting from a cell fusion are not “recombinant polypeptides.” Further, viral proteins produced by a cell as a result of viral infection with a naturally-occurring virus are also not “recombinant polypeptides” as meant herein unless the viral nucleic acid has been altered by genetic engineering prior to infecting the cell.

“Substantially similar” polypeptides are at least 80%, optionally at least 90%, identical to each other in amino acid sequence and maintain or alter in a desirable manner the biological activity of the unaltered polypeptide. Conservative amino acid substitutions, unlikely to affect biological activity, include, without limitation, the following: Ala for Ser, Val for Ile, Asp for Glu, Thr for Ser, Ala for Gly, Ala for Thr, Ser for Asn, Ala for Val, Ser for Gly, Tyr for Phe, Ala for Pro, Lys for Arg, Asp for Asn, Leu for Ile, Leu for Val, Ala for Glu, Asp for Gly, and these changes in the reverse. See e.g. Neurath et al., The Proteins, Academic Press, New York (1979). In addition, exchanges of amino acids among members of the following six groups of amino acids will be considered to be conservative substitutions for the purposes of the invention. The groups are: 1) methionine, alanine, valine, leucine, and isoleucine; 2) cysteine, serine, threonine, asparagine, and glutamine; 3) aspartate and glutamate; 4) histidine, lysine, and arginine; 5) glycine and proline; and 6) tryptophan, tyrosine, and phenylalanine. The percent identity of two amino sequences can be determined by visual inspection and mathematical calculation, or more preferably, the comparison is done by comparing sequence information using a computer program such as the Genetics Computer Group (GCG; Madison, Wis.) Wisconsin package version 10.0 program, ‘GAP’ (Devereux et al. (1984), Nucl. Acids Res. 12: 387) or other comparable computer programs. The preferred default parameters for the ‘GAP’ program includes: (1) the weighted amino acid comparison matrix of Gribskov and Burgess (1986), Nucl. Acids Res. 14: 6745, as described by Schwartz and Dayhoff, eds., Atlas of polypeptide Sequence and Structure, National Biomedical Research Foundation, pp. 353-358 (1979), or other comparable comparison matrices; (2) a penalty of 30 for each gap and an additional penalty of 1 for each symbol in each gap for amino acid sequences; (3) no penalty for end gaps; and (4) no maximum penalty for long gaps. Other programs used by those skilled in the art of sequence comparison can also be used.

“Transition phase” means a period of cell culture between a “growth phase” and a “production phase.” During transition phase, the medium and environmental conditions are typically shifted from those designed to maximize proliferation to those designed to maximize polypeptide production.

A “variable antibody immunoglobulin domain” is an immunoglobulin domain that is identical or substantially similar to a V_{L} or a V_{H} domain of human or animal origin.

The present invention is directed towards improved methods for culturing mammalian cells, which may have been genetically engineered to produce a particular polypeptide. In particular, the invention is directed towards culture methods that maximize the production of specific polypeptides. It is also directed towards methods of producing and obtaining such polypeptides from cultured mammalian cells. Polypeptides are useful in a large variety of diagnostic, therapeutic, agricultural, nutritional, and research applications.

As shown by the experimental data reported herein, it has been discovered that an aromatic carboxylic acid, an acetamide, and a hydroxamic acid compound used separately or in various combinations can induce dramatically the production of recombinant polypeptide from CHO cell lines. These compounds were first identified as inducers in a 96-well fluorescent protein based assay. Using a construct that expressed DsRed under the control of a EASE/CMV promoter, including an adenoviral tripartite leader, as an indicator for expression, various compounds were assayed. Those compounds that appeared to induce DsRed expression, especially at lowered temperatures, were then further investigated in assays for induction of other recombinant polypeptides. These experiments led to the identification of a subset of chemicals as strong inducers of recombinant protein expression. Generally, the compounds fell into three broad classes: aromatic carboxylic acid, acetamide, and hydroxamic acid compounds. Additional experiments revealed that combinations of compounds from more than one of these classes could further increase induction of polypeptide expression, especially recombinant protein production. Thus, the use of these compounds as inducers can substantially reduce manufacturing costs and/or decrease plant capacity needs.

An aromatic carboxylic acid useful in the practice of the invention is a compound of the formula X-Y-Z, wherein X is an aromatic group, for example, a 5, 6, or 7 membered carbon ring, Y is a connector with an alkyl group of from 1 to 20 or more carbons, preferably 2 to 10 carbons, more preferably 2-5 carbons, and Z is a carboxylic acid group. The aromatic group can be substituted or not substituted; if a substituted phenyl group is used, the substitution is preferably at the para position, although meta and ortho substituents can be tolerated. The aromatic group appears to be particularly advantageous; it has been found that if this group is substituted with a straight or branch chain alkyl such compounds do not work nearly as well, and that dye groups or tri-acids are negative. For example, pimelic acid, methylsuccinic acid, and sodium dihydrogen citrate were ineffective as inducers. Illustrative examples of these aromatic carboxylic acids whose usefulness as inducers of recombinant polypeptide production is described herein are as follows:

Aromatic carboxylic acid class:

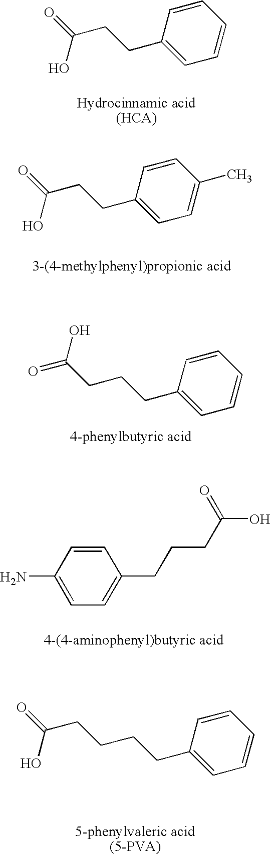

Other compounds that can be used to increase polypeptide production are: 3-(4-hydroxyphenyl)propionic acid; 3-(2-methylphenyl)propionic acid; 4-(4-methoxyphenyl)butyric acid; 4-(4-aminophenyl)butyric acid; 3-(2-hydroxyphenyl)propionic acid; 6-phenylhexanoic acid; 3,4-difluorohydocinnamic acid; and 2-methylindole-3-acetic acid. Still other compounds that can be used are: 3-(3-methoxyphenyl)propionic acid; 6-benzyloxycarbonylaminohexanoic acid; 3-[4-(trifluoromethyl)phenyl]propionic acid; 3-(4-aminophenyl)propionic acid; 3-(4-fluorophenyl)propionic acid; 2-thienylacetic acid, and 3-(3,4-dimethoxyphenyl)propionic acid.

In addition, the invention encompasses the use of acetamides as inducers of polypeptide production. Copending patent application Ser. No. 10/400,334 describes the use of hybrid polar compounds, some of which are acetamides, as inducers. However, as described herein, acetamides that are not hybrid polar compounds (i.e., contain only one polar group—the acetamide group) can also induce recombinant polypeptide production. Such non-hybrid polar acetamides can be alkyl acetamides wherein the alkyl chain is from about 32 to about 20 carbons in length. Examples of acetamides that can be used alone or in combination with other compounds as inducers include the following.

Acetamide class:

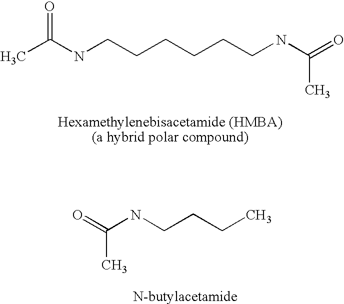

Further, another class of compounds which can be used as inducers of recombinant polypeptide production are hydroxamic acids. The invention encompasses the use as inducers of hydroxamic acids which are not hybrid polar compounds. Examples of compounds shown herein to be useful are as follows:

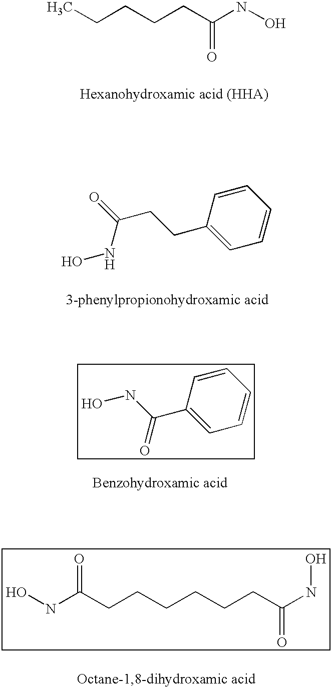

In particular, it has been found through screening a large number of different compounds that addition of any of the above exemplary aromatic carboxylic acid, acetamide, and hydroxamic acid compounds to the production phase of a cell culture can enhance recombinant polypeptide production. Further, such compounds chosen from more than one of the above classes can be added in combination to enhance recombinant polypeptide production.

Furthermore, other methods of increasing production, such as, for example, culturing the cells at temperatures from about 29° C. to about 36° C., between about 29° C. and 35° C., and/or from about 30° C. to about 33° C. can also be used in combination with one or more of these chemical inducers. Optionally, cell culture using the methods of the invention can take place during a production phase, as distinguished from a growth phase. A growth phase can be distinguished from a production phase by, for example, a temperature shift and/or a change in medium such as, for example, the addition of one or more inducers.

In one aspect, the invention provides a method comprising growing in culture a mammalian cell that has been genetically engineered to produce a polypeptide; and adding to the culture one or more of an aromatic carboxylic acid, an acetamide, and a hydroxamic acid compound. A genetically engineered cell may be a cell that has been transformed with a recombinant vector encoding the polypeptide. In addition, the polypeptide can be expressed under the control of a heterologous promoter such as, for example, a CMV promoter or a SV40 promoter. Typically, the cell does not naturally express the polypeptide or only naturally expresses the polypeptide at very low levels (in the absence of genetic engineering). In another aspect, the invention provides a culture containing a cell genetically engineered to produce a polypeptide, a production medium, and an aromatic carboxylic acid, an acetamide, and/or a hydroxamic acid compound.

In addition, the methods and compositions of the invention can be used in combination with any other known or yet to be discovered methods of inducing the production of recombinant polypeptides. Such techniques include cold temperature shift, alkanoic acid additions (as described in U.S. Pat. No. 5,705,364 to Etcheverry et al., incorporated herein by reference), hybrid dipolar compounds, xanthines, DMF, and DMSO, to name just a few examples, as well as any yet to be described and/or discovered induction techniques (see, for example, copending patent application Ser. No. 10/400,334, filed Mar. 27, 2003, incorporated by reference herein). As used herein, “inducing” polypeptide production or “induction” refers to culturing cells under a set of conditions designed to maximize the total amount of a desired polypeptide made by the cells. An “inducer” is an agent that, when added to culture medium, can increase the production of a desired polypeptide in at least some cell lines.

Combining the addition of an aromatic carboxylic acid, an acetamide, and/or a hydroxamic acid compound with one another and/or with other protein induction techniques can have a synergistic effect on polypeptide induction, allowing for lower additions of these compounds and/or lower additions of other inducing agents and/or more conservative temperature shifts. The other methods of induction can take place at around the same time as the compound is added, and/or before and/or after addition. For example, one can shift the temperature of the culture at day 0, and then add one of these compounds, and optionally other chemical inducers, later, e.g. one to several hours or days later. Such a protocol allows some additional growth of a seeded culture before full induction. Furthermore, multiple additions of an aromatic carboxylic acid, an acetamide, and/or a hydroxamic acid compound can be added to the culture during the production phase, separated by about 12, 24, 48, and/or 72 hours or more, with or without additions of other inducing agents or changes in culture conditions. For example, an inducer can be added at day 0 and again at day 4. Alternatively, an inducer can be added for the first time one, two, three, or four days after a temperature shift.

In one aspect, the invention entails performing a low temperature shift (shifting the temperature of the medium from the optimal growth temperature, usually around 37° C., to a lower temperature, usually from about 29° C. to about 36° C., and optionally about 30° C. to about 34° C. at the time of, before, and/or after adding the inducer compound or compounds.

There are individual differences between cell lines in the effectiveness of various inducers. For example, although sodium butyrate is a widely-used inducer, it can have no effect or an adverse effect on polypeptide production in some cell lines. Different inducers or different concentrations of the same inducers may be appropriate for different cell lines. Furthermore, different temperatures may be appropriate for different cell lines. In spite of this variability, inducers such as aromatic carboxylic acids, acetamides and hydroxamic acids can be useful in a wide variety of cell lines.

The optimal concentration for a particular compound will vary depending on its activity and the cell line in which it is used and can be determined by one skilled in the art using routine methods and the guidance provided herein. For example, compounds such as hydrocinnamic acid (HCA), 3-(4-methylphenyl)propionic acid, 4-phenylbutyric acid, 4-(4-aminophenyl)butyric acid, and 5-phenylvaleric acid can be added at concentrations from about 0.01 millimolar to about 20 millimolar, preferably between about 0.1 millimolar and about 5 millimolar, and more preferably at about 0.2 to 2 millimolar. Compounds such as hexanohydroxamic acid (HHA) and 3-phenylpropionohydroxamic acid should be used at somewhat lower concentrations and thus can be added at concentrations from about 0.01 micromolar to about 1 millimolar, preferably between about 0.1 micromolar and about 50 micromolar, and more preferably at about 1 to 20 micromolar.

Particularly preferred polypeptides for expression are polypeptide-based drugs, also known as biologics. Preferably, the polypeptides are secreted as extracellular products. The polypeptide being produced can comprise part or all of a polypeptide that is identical or substantially similar to a naturally-occurring polypeptide, and/or it may, or may not, be a recombinant fusion polypeptide. Optionally, the polypeptide may be a human polypeptide, a fragment thereof, or a substantially similar polypeptide that is at least 15 amino acids in length. It may comprise a non-antibody polypeptide and/or an antibody. It may be produced intracellularly or be secreted into the culture medium from which it can be recovered. It may or may not be a soluble polypeptide.

The polypeptide being produced can comprise part or all of a polypeptide that is identical or substantially similar to a naturally-occurring polypeptide, and/or it may, or may not, be a recombinant fusion polypeptide. It may comprise a non-antibody polypeptide and/or an antibody. It may be produced intracellularly or be secreted into the culture medium from which it can be recovered.

The invention can be used to induce the production of just about any polypeptide, and is particularly advantageous for polypeptides whose expression is under the control of a strong promoter, such as for example, a viral promoter, and/or polypeptides that are encoded on a message that has an adenoviral tripartite leader element. Examples of useful expression vectors that can be used to produce proteins are disclosed in International Application WO 01/27299 and in McMahan et al., (1991), EMBO J. 10: 2821, which describes the pDC409 vector, which uses one viral promoter, a CMV promoter. A protein is generally understood to be a polypeptide of at least about 10 amino acids, optionally about 25, 75, or 100 amino acids.

Generally, the methods of the invention are useful for inducing the production of recombinant polypeptides. Some polypeptides that can be produced with the methods of the invention include polypeptides comprising amino acid sequences identical to or substantially similar to all or part of one of the following polypeptides: a flt3 ligand (as described in International Application WO 94/28391, incorporarted herein by reference), a CD40 ligand (as described in U.S. Pat. No. 6,087,329 incorporated herein by reference), erythropoeitin, thrombopoeitin, calcitonin, leptin, IL-2, angiopoietin-2 (as described by Maisonpierre et al. (1997), Science 277(5322): 55-60, incorporated herein by reference), Fas ligand, ligand for receptor activator of NF-kappa B (RANKL, as described in International Application WO 01/36637, incorporated herein by reference), tumor necrosis factor (TNF)-related apoptosis-inducing ligand (TRAIL, as described in International Application WO 97/01633, incorporated herein by reference), thymic stroma-derived lymphopoietin, granulocyte colony stimulating factor, granulocyte-macrophage colony stimulating factor (GM-CSF, as described in Australian Patent No. 588819, incorporated herein by reference), mast cell growth factor, stem cell growth factor (described in e.g. U.S. Pat. No. 6,204,363, incorporated herein by reference), epidermal growth factor, keratinocyte growth factor, megakaryote growth and development factor, RANTES, growth hormone, insulin, insulinotropin, insulin-like growth factors, parathyroid hormone, interferons including a interferons, y interferon, and consensus interferons (such as those described in U.S. Pat. Nos. 4,695,623 and 4,897471, both of which are incorporated herein by reference), nerve growth factor, brain-derived neurotrophic factor, synaptotagmin-like proteins (SLP 1-5), neurotrophin-3, glucagon, interleukins 1 through 18, colony stimulating factors, lymphotoxin-β, tumor necrosis factor (TNF), leukemia inhibitory factor, oncostatin-M, and various ligands for cell surface molecules ELK and Hek (such as the ligands for eph-related kinases or LERKS). Descriptions of polypeptides that can be produced according to the inventive methods may be found in, for example, Human Cytokines: Handbook for Basic and Clinical Research, Vol. II (Aggarwal and Gutterman, eds. Blackwell Sciences, Cambridge, Mass., 1998); Growth Factors: A Practical Approach (McKay and Leigh, eds., Oxford University Press Inc., New York, 1993); and The Cytokine Handbook (A. W. Thompson, ed., Academic Press, San Diego, Calif., 1991), all of which are incorporated herein by reference.

Other polypeptides that can be produced using the methods of the invention include polypeptides comprising all or part of the amino acid sequence of a receptor for any of the above-mentioned polypeptides, an antagonist to such a receptor or any of the above-mentioned polypeptides, and/or polypeptides substantially similar to such receptors or antagonists. These receptors and antagonists include: both forms of tumor necrosis factor receptor (TNFR, referred to as p55 and p75, as described in U.S. Pat. No. 5,395,760 and U.S. Pat. No. 5,610,279, both of which are incorporated herein by reference), Interleukin-1 (IL-1) receptors (types I and II; described in EP Patent No. 0 460 846, U.S. Pat. No. 4,968,607, and U.S. Pat. No. 5,767,064, all of which are incorporated herein by reference), IL-1 receptor antagonists (such as those described in U.S. Pat. No. 6,337,072, incorporated herein by reference), IL-1 antagonists or inhibitors (such as those described in U.S. Pat. Nos. 5,981,713, 6,096,728, and 5,075,222, all of which are incorporated herein by reference) IL-2 receptors, IL-4 receptors (as described in EP Patent No. 0 367 566 and U.S. Pat. No. 5,856,296, both of which are incorporated by reference), IL-15 receptors, IL-17 receptors, IL-18 receptors, granulocyte-macrophage colony stimulating factor receptor, granulocyte colony stimulating factor receptor, receptors for oncostatin-M and leukemia inhibitory factor, receptor activator of NF-kappa B (RANK, described in WO 01/36637 and U.S. Pat. No. 6,271,349, both of which are incorporated by reference), osteoprotegerin (described in e.g. U.S. Pat. No. 6,015,938, incorporated by reference), receptors for TRAIL (including TRAIL receptors 1, 2, 3, and 4), and receptors that comprise death domains, such as Fas or Apoptosis-Inducing Receptor (AIR).

Other polypeptides that can be produced using the process of the invention include polypeptides comprising all or part of the amino acid sequences of differentiation antigens (referred to as CD polypeptides) or their ligands or polypeptides substantially similar to either of these. Such antigens are disclosed in Leukocyte Typing VI (Proceedings of the VIth International Workshop and Conference, Kishimoto, Kikutani et al., eds., Kobe, Japan, 1996, which is incorporated by reference). Similar CD polypeptides are disclosed in subsequent workshops. Examples of such antigens include CD22, CD27, CD30, CD39, CD40, and ligands thereto (CD27 ligand, CD30 ligand, etc.). Several of the CD antigens are members of the TNF receptor family, which also includes 41BB and OX40. The ligands are often members of the TNF family, as are 41BB ligand and OX40 ligand. Accordingly, members of the TNF and TNFR families can also be purified using the present invention.

Enzymatically active polypeptides or their ligands can also be produced according to the methods of the invention. Examples include polypeptides comprising all or part of one of the following polypeptides or their ligands or a polypeptide substantially similar to one of these: metalloproteinase-disintegrin family members, various kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, TNF-alpha Converting Enzyme, ligands for any of the above-mentioned enzymes, and numerous other enzymes and their ligands.

The methods of the invention can also be used to produce antibodies or portions thereof and chimeric antibodies, i.e. antibodies having human constant antibody immunoglobulin domains coupled to one or more murine variable antibody immunoglobulin domain, fragments thereof, or substantially similar proteins. The methods of the invention may also be used to produce conjugates comprising an antibody and a cytotoxic or luminescent substance. Such substances include: maytansine derivatives (such as DM1); enterotoxins (such as a Staphlyococcal enterotoxin); iodine isotopes (such as iodine-125); technium isotopes (such as Tc-99m); cyanine fluorochromes (such as Cy5.5.18); and ribosome-inactivating polypeptides (such as bouganin, gelonin, or saporin-S6). The invention can also be used to produce chimeric proteins selected in vitro to bind to a specific target protein and modify its activity such as those described in International Applications WO 01/83525 and WO 00/24782, both of which are incorporated by reference. Examples of antibodies, in vitro-selected chimeric proteins, or antibody/cytotoxin or antibody/luminophore conjugates that can be produced by the methods of the invention include those that recognize any one or a combination of polypeptides including, but not limited to, the above-mentioned proteins and/or the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-1α, IL-1β, IL-2, IL-3, IL-7, IL-4, IL-5, IL-8, IL-10, IL-2 receptor, IL-4 receptor, IL-6 receptor, IL-13 receptor, IL-18 receptor subunits, PDGF-β and analogs thereof (such as those described in U.S. Pat. Nos. 5,272,064 and 5,149,792), VEGF, TGF, TGF-β2, TGF-β1, EGF receptor (including those described in U.S. Pat. No. 6,235,883 B1, incorporated by reference) VEGF receptor, hepatocyte growth factor, osteoprotegerin ligand, interferon gamma, B lymphocyte stimulator (BlyS, also known as BAFF, THANK, TALL-1, and zTNF4; see Do and Chen-Kiang (2002), Cytokine Growth Factor Rev. 13(1): 19-25), C5 complement, IgE, tumor antigen CA125, tumor antigen MUC1, PEM antigen, LCG (which is a gene product that is expressed in association with lung cancer), HER-2, a tumor-associated glycoprotein TAG-72, the SK-1 antigen, tumor-associated epitopes that are present in elevated levels in the sera of patients with colon and/or pancreatic cancer, cancer-associated epitopes or polypeptides expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, B2 integrins, TRAIL receptors 1, 2, 3, and 4, RANK, RANK ligand, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA-4 (which is a cytotoxic T lymphocyte-associated antigen), Fc-γ-1 receptor, HLA-DR 10 beta, HLA-DR antigen, L-selectin, Respiratory Syncitial Virus, human immunodeficiency virus (HIV), hepatitis B virus (HBV), Streptococcus mutans, and Staphlycoccus aureus.

The invention may also be used to produce all or part of an anti-idiotypic antibody or a substantially similar polypeptide, including anti-idiotypic antibodies against: an antibody targeted to the tumor antigen gp72; an antibody against the ganglioside GD3; an antibody against the ganglioside GD2; or antibodies substantially similar to these.

The methods of the invention can also be used to produce recombinant fusion polypeptides comprising any of the above-mentioned polypeptides. For example, recombinant fusion polypeptides comprising one of the above-mentioned polypeptides plus a multimerization domain, such as a leucine zipper, a coiled coil, an Fc portion of an antibody, or a substantially similar protein, can be produced using the methods of the invention. See e.g. WO94/10308; Lovejoy et al. (1993), Science 259:1288-1293; Harbury et al. (1993), Science 262:1401-05; Harbury et al. (1994), Nature 371:80-83; H\dot{a}kansson et al. (1999), Structure 7:255-64, all of which are incorporated by reference. Specifically included among such recombinant fusion polypeptides are polypeptides in which a portion of TNFR or RANK is fused to an Fc portion of an antibody (TNFR:Fc or RANK:Fc). TNFR:Fc comprises the Fc portion of an antibody fused to an extracellular domain of TNFR, which includes amino acid sequences substantially similar to amino acids 1-163, 1-185, or 1-235 of FIG. 2A of U.S. Pat. No. 5,395,760, which is incorporated by reference. RANK:Fc is described in International Application WO 01/36637, which is incorporated by reference.

Preferably, the polypeptides are expressed under the control of a heterologous control element such as, for example, a promoter that does not in nature direct the production of that polypeptide. For example, the promoter can be a strong viral promoter (e.g., CMV, SV40) that directs the expression of a mammalian polypeptide. The host cell may or may not normally produce the polypeptide. For example, the host cell can be a CHO cell that has been genetically engineered to produce a human polypeptide, meaning that nucleic acid encoding the human polypeptide has been introduced into the CHO cell. Alternatively, the host cell can be a human cell that has been genetically engineered to produce increased levels of a human polypeptide normally present only at very low levels (e.g., by replacing the endogenous promoter with a strong viral promoter). For the production of recombinant polypeptides, an expression vector encoding the recombinant polypeptide can be transferred, for example by transfection or viral infection, into a substantially homogeneous culture of host cells. The expression vector, which can be constructed using the methods of genetic engineering, can include nucleic acids encoding the polypeptide of interest operably linked to suitable regulatory sequences.

The regulatory sequences are typically derived from mammalian, microbial, viral, and/or insect genes. Examples of regulatory sequences include transcriptional promoters, operators, and enhancers, a ribosomal binding site (see e.g. Kozak (1991), J. Biol. Chem. 266:19867-19870), appropriate sequences to control transcriptional and translational initiation and termination, polyadenylation signals (see e.g. McLauchlan et al. (1988), Nucleic Acids Res. 16:5323-33), and matrix and scaffold attachment sites (see Phi-Van et al. (1988), Mol. Cell. Biol. 10:2302-07; Stief et al. (1989), Nature 341:342-35; Bonifer et al. (1990), EMBO J. 9:2843-38). Nucleotide sequences are operably linked when the regulatory sequence functionally relates to the polypeptide coding sequence. Thus, a promoter nucleotide sequence is operably linked to a polypeptide coding sequence if the promoter nucleotide sequence controls the transcription of the coding sequence. A gene encoding a selectable marker is generally incorporated into the expression vector to facilitate the identification of recombinant cells.

Transcriptional and translational control sequences for mammalian host cell expression vectors can be excised from viral genomes. Commonly used promoter and enhancer sequences are derived from polyoma virus, adenovirus 2, simian virus 40 (SV40), and human cytomegalovirus (CMV). For example, the human CMV promoter/enhancer of immediate early gene 1 may be used. See e.g. Patterson et al. (1994), Applied Microbiol. Biotechnol. 40:691-98. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al. (1978), Nature 273:113; Kaufman (1990), Meth. in Enzymol. 185:487-511). Smaller or larger SV40 fragments can also be used, provided the approximately 250 bp sequence extending from the Hind III site toward the Bgl I site located in the SV40 viral origin of replication site is included.

In addition, a sequence encoding an appropriate native or heterologous signal peptide (leader sequence) can be incorporated into the expression vector, to promote extracellular secretion of the recombinant polypeptide. The signal peptide will be cleaved from the recombinant polypeptide upon secretion from the cell. The choice of signal peptide or leader depends on the type of host cells in which the recombinant polypeptide is to be produced. Examples of signal peptides that are functional in mammalian host cells include the signal sequence for interleukin-7 (IL-7) described in U.S. Pat. No. 4,965,195, the signal sequence for interleukin-2 receptor described in Cosman et al. (1984), Nature 312:768; the interleukin-4 receptor signal peptide described in EP Patent No. 367,566; the type I interleukin-1 receptor signal peptide described in U.S. Pat. No. 4,968,607; and the type II interleukin-1 receptor signal peptide described in EP Patent No. 0 460 846.

Established methods for introducing DNA into mammalian cells have been described. Kaufman, R. J., Large Scale Mammalian Cell Culture, 1990, pp. 15-69. Additional protocols using commercially available reagents, such as the cationic lipid reagents LIPOFECTAMINE™, LIPOFECTAMINE™-2000, or LIPOFECTAMINE™-PLUS (which can be purchased from Invitrogen), can be used to transfect cells. Felgner et al. (1987)., Proc. Natl. Acad. Sci. USA 84:7413-7417. In addition, electroporation or bombardment with microprojectiles coated with nucleic acids can be used to transfect mammalian cells using procedures, such as those in Sambrook et al., Molecular Cloning: A Laboratory Manual, 2nd ed. Vol. 1-3, Cold Spring Harbor Laboratory Press (1989) and Fitzpatrick-McElligott (1992), Biotechnology (NY) 10(9):1036-40. Selection of stable transformants can be performed using methods known in the art, such as, for example, resistance to cytotoxic drugs. Generally, in mammalian host cells stable transformants have the introduced polynucleotides incorporated into the chromosome. Kaufman et al. ((1990), Meth. in Enzymology 185:487-511), describes several selection schemes, such as dihydrofolate reductase (DHFR) resistance. A suitable host strain for DHFR selection can be CHO strain DX-B11, which is deficient in DHFR. Urlaub and Chasin (1980), Proc. Natl. Acad. Sci. USA 77:4216-4220. A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Additional control sequences shown to improve expression of heterologous genes from mammalian expression vectors include such elements as the expression augmenting sequence element (EASE) derived from CHO cells (Morris et al., in Animal Cell Technology, pp. 529-534 (1997); U.S. Pat. Nos. 6,312,951 B1, 6,027,915, and 6,309,841 B1) and the tripartite leader (TPL) and VA gene RNAs from Adenovirus 2 (Gingeras et al. (1982), J. Biol. Chem. 257:13475-13491). The internal ribosome entry site (IRES) sequences of viral origin allows dicistronic mRNAs to be translated efficiently (Oh and Sarnow (1993), Current Opinion in Genetics and Development 3:295-300; Ramesh et al. (1996), Nucleic Acids Research 24:2697-2700). Expression of a heterologous cDNA as part of a dicistronic mRNA followed by the gene for a selectable marker (e.g. DHFR) has been shown to improve transfectability of the host and expression of the heterologous cDNA (Kaufman et al. (1990), Methods in Enzymol. 185:487-511). Exemplary expression vectors that employ dicistronic mRNAs are pTR-DC/GFP described by Mosser et al., Biotechniques 22:150-161 (1997), and p2A5I described by Morris et al., in Animal Cell Technology, pp. 529-534 (1997).

A useful high expression vector, pCAVNOT, has been described by Mosley et al. ((1989), Cell 59:335-348). Other expression vectors for use in mammalian host cells can be constructed as disclosed by Okayama and Berg ((1983), Mol. Cell. Biol. 3:280). A useful system for stable high level expression of mammalian cDNAs in C127 murine mammary epithelial cells can be constructed substantially as described by Cosman et al. ((1986), Mol. Immunol. 23:935). A useful high expression vector, PMLSV N1/N4, described by Cosman et al. ((1984), Nature 312:768), has been deposited as ATCC 39890. Additional useful mammalian expression vectors are described in EP Patent No.-A-0 367 566 and WO 01/27299 A1. The vectors can be derived from retroviruses. In place of the native signal sequence, a heterologous signal sequence can be added, such as one of the following sequences: the signal sequence for IL-7 described in U.S. Pat. No. 4,965,195; the signal sequence for IL-2 receptor described in Cosman et al. (Nature 312:768 (1984)); the IL-4 signal peptide described in EP Patent No. 0 367 566; the type I IL-1 receptor signal peptide described in U.S. Pat. No. 4,968,607; and the type II IL-1 receptor signal peptide described in EP Patent No. 0 460 846.

The polypeptides can be produced recombinantly in eukaryotic cells and are preferably secreted by host cells adapted to grow in cell culture. Optionally, host cells for use in the invention are preferably mammalian cells. The cells can be also genetically engineered to express a gene of interest, can be mammalian production cells adapted to grow in cell culture, and/or can be homogenous cell lines. Examples of such cells commonly used in the industry are VERO, BHK, HeLa, CV1 (including Cos), MDCK, 293, 3T3, myeloma cell lines (e.g., NSO, NS1), PC12, WI38 cells, and Chinese hamster ovary (CHO) cells, which are widely used for the production of several complex recombinant polypeptides, e.g. cytokines, clotting factors, and antibodies (Brasel et al. (1996), Blood 88:2004-2012; Kaufman et al. (1988), J. Biol Chem 263:6352-6362; McKinnon et al. (1991), J Mol Endocrinol 6:231-239; Wood et al. (1990), J. Immunol. 145:3011-3016). The dihydrofolate reductase (DHFR)-deficient mutant cell lines (Urlaub et al. (1980), Proc Natl Acad Sci USA 77: 4216-4220, which is incorporated by reference), DXB11 and DG44, are desirable CHO host cell lines because the efficient DHFR selectable and amplifiable gene expression system allows high level recombinant polypeptide expression in these cells (Kaufman R. J. (1990), Meth Enzymol 185:537-566, which is incorporated by reference). In addition, these cells are easy to manipulate as adherent or suspension cultures and exhibit relatively good genetic stability. CHO cells and recombinant polypeptides expressed in them have been extensively characterized and have been approved for use in clinical commercial manufacturing by regulatory agencies. The methods of the invention can also be practiced using hybridoma cell lines that produce an antibody. Methods for making hybridoma lines are well known in the art. See e.g. Berzofsky et al. in Paul, ed., Fundamental Immunology, Second Edition, pp.315-356, at 347-350, Raven Press Ltd., New York (1989). Cell lines derived from the above-mentioned lines are also suitable for practicing the invention.

According to the present invention, a mammalian host cell is cultured under conditions that promote the production of the polypeptide of interest, which can be an antibody or a recombinant polypeptide. Basal cell culture medium formulations are well known in the art. To these basal culture medium formulations the skilled artisan will add components such as amino acids, salts, sugars, vitamins, hormones, growth factors, buffers, antibiotics, lipids, trace elements and the like, depending on the requirements of the host cells to be cultured. The culture medium may or may not contain serum and/or protein. Various tissue culture media, including serum-free and/or defined culture media, are commercially available for cell culture. Tissue culture media is defined, for purposes of the invention, as a media suitable for growth of animal cells, and preferably mammalian cells, in vitro cell culture. Typically, tissue culture media contains a buffer, salts, energy source, amino acids, vitamins and trace essential elements. Any media capable of supporting growth of the appropriate eukaryotic cell in culture can be used; the invention is broadly applicable to eukaryotic cells in culture, particularly mammalian cells, and the choice of media is not crucial to the invention. Tissue culture media suitable for use in the invention are commercially available from, e.g., ATCC (Manassas, Va.). For example, any one or combination of the following media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (available from JRH Biosciences, Lenexa, Kans., USA), among others, which can be obtained from the American Type Culture Collection or JRH Biosciences, as well as other vendors. When defined medium that is serum-free and/or peptone-free is used, the medium is usually highly enriched for amino acids and trace elements. See, for example, U.S. Pat. Nos. 5,122,469 to Mather et al. and 5,633,162 to Keen et al.

In the methods and compositions of the invention, cells can be grown in serum-free, protein-free, growth factor-free, and/or peptone-free media. The term “serum-free” as applied to media includes any mammalian cell culture medium that does not contain serum, such as fetal bovine serum. The term “insulin-free” as applied to media includes any medium to which no exogenous insulin has been added. By exogenous is meant, in this context, other than that produced by the culturing of the cells themselves. The term “IGF-1-free” as applied to media includes any medium to which no exogenous Insulin-like growth factor-1 (IGF-1) or analog (such as, for example, LongR3, [Ala31], or [Leu24][Ala31] IGF-1 analogs available from GroPep Ltd. of Thebarton, South Australia) has been added. The term “growth-factor free” as applied to media includes any medium to which no exogenous growth factor (e.g., insulin, IGF-1) has been added. The term “protein-free” as applied to media includes medium free from exogenously added protein, such as, for example, transferring and the protein growth factors IGF-1 and insulin. Protein-free media may or may not have peptones. The term “peptone-free” as applied to media includes any medium to which no exogenous protein hydrolysates have been added such as, for example, animal and/or plant protein hydrolysates. Eliminating peptone from media has the advantages of reducing lot to lot variability and enhancing processing such as filtration. Chemically defined media are media in which every component is defined and obtained from a pure source, preferably a non-animal source.

The skilled artisan may also choose to use one of the many individualized media formulations that have been developed to maximize cell growth, cell viability, and/or recombinant polypeptide production in a particular cultured host cell. The methods according to the current invention may be used in combination with commercially available cell culture media or with a cell culture medium that has been individually formulated for use with a particular cell line. For example, an enriched medium that could support increased polypeptide production may comprise a mixture of two or more commercial media, such as, for instance, DMEM and Ham's F12 media combined in ratios such as, for example, 1:1, 1:2, 1:3, 1:4, 1:5, 1:6, 1:7, 1:8, or even up to 1:15 or higher. Alternatively or in addition, a medium can be enriched by the addition of nutrients, such as amino acids or peptone, and/or a medium (or most of its components with the exceptions noted below) can be used at greater than its usual, recommended concentration, for example at 2×, 3×, 4×, 5×, 6×, 7×, 8×, or even higher concentrations. As used herein, “1×” means the standard concentration, “2×” means twice the standard concentration, etc. In any of these embodiments, medium components that can substantially affect osmolarity, such as salts, cannot be increased in concentration so that the osmolarity of the medium falls outside of an acceptable range. Thus, a medium may, for example, be 8× with respect to all components except salts, which can be present at only 1×. An enriched medium may be serum free and/or protein free. Further, a medium may be supplemented periodically during the time a culture is maintained to replenish medium components that can become depleted such as, for example, vitamins, amino acids, and metabolic precursors. As is known in the art, different media and temperatures may have somewhat different effects on different cell lines, and the same medium and temperature may not be suitable for all cell lines.

Suitable culture conditions for mammalian cells are known in the art. See e.g. Animal cell culture: A Practical Approach, D. Rickwood, ed., Oxford University Press, New York (1992). Mammalian cells may be cultured in suspension or while attached to a solid substrate. Furthermore, mammalian cells may be cultured, for example, in fluidized bed bioreactors, hollow fiber bioreactors, roller bottles, shake flasks, or stirred tank bioreactors, with or without microcarriers, and operated in a batch, fed batch, continuous, semi-continuous, or perfusion mode.

The methods according to the present invention may be used to improve the production of recombinant polypeptides in both single phase and multiple phase culture processes. In a single phase process, cells are inoculated into a culture environment and the disclosed methods are employed during the single production phase. In a multiple stage process, cells are cultured in two or more distinct phases. For example cells may be cultured first in a growth phase, under environmental conditions that maximize cell proliferation and viability, then transferred to a production phase, under conditions that maximize polypeptide production. The growth and production phases may be preceded by, or separated by, one or more transition phases. In multiple phase processes the methods according to the present invention are employed at least during the production phase. A growth phase may occur at a higher temperature than a production phase. For example, a growth phase may occur at a first temperature from about 35° C. to about 38° C., and a production phase may occur at a second temperature from about 29° C. to about 36° C., optionally from about 30° C. to about 33° C. Chemical inducers of polypeptide production, such as, for example, aromatic carboxylic acids, acetamides, and/or hydroxamic acids (as well as, optionally, other inducers) may be added at the same time as, before, and/or after a temperature shift. If inducers are added after a temperature shift, they can be added from one hour to five days after the temperature shift, optionally from one to two days after the temperature shift.

After induction using the methods of the invention, the resulting expressed polypeptide can then be collected. In addition, the polypeptide can purified, or partially purified, from such culture or component (e.g., from culture medium or cell extracts or bodily fluid) using known processes. By “partially purified” means that some fractionation procedure, or procedures, have been carried out, but that more polypeptide species (at least 10%) than the desired polypeptide is present. By “purified” is meant that the polypeptide is essentially homogeneous, i.e., less than 1% contaminating polypeptides are present. Fractionation procedures can include but are not limited to one or more steps of filtration, centrifugation, precipitation, phase separation, affinity purification, gel filtration, ion exchange chromatography, hydrophobic interaction chromatography (HIC; using such resins as phenyl ether, butyl ether, or propyl ether), HPLC, or some combination of above.

For example, the purification of the polypeptide can include an affinity column containing agents which will bind to the polypeptide; one or more column steps over such affinity resins as concanavalin A-agarose, heparin-TOYOPEARL® (Toyo Soda Manufacturing Co., Ltd., Japan) or Cibacrom blue 3GA SEPHAROSE® (Pharmacia Fine Chemicals, Inc., New York); one or more steps involving elution; and/or immunoaffinity chromatography. The polypeptide can be expressed in a form that facilitates purification. For example, it may be expressed as a fusion polypeptide, such as those of maltose binding polypeptide (MBP), glutathione-S-transferase (GST), or thioredoxin (TRX). Kits for expression and purification of such fusion polypeptides are commercially available from New England BioLab (Beverly, Mass.), Pharmacia (Piscataway, N.J.) and InVitrogen, respectively. The polypeptide can be tagged with an epitope and subsequently purified by using a specific antibody directed to such epitope. One such epitope (FLAG®) is commercially available from Kodak (New Haven, Conn.). It is also possible to utilize an affinity column comprising a polypeptide-binding protein, such as a monoclonal antibody to the recombinant polypeptide, to affinity-purify expressed polypeptides. Other types of affinity purification steps can be a Protein A or a Protein G column, which affinity agents bind to proteins that contain Fc domains. Polypeptides can be removed from an affinity column using conventional techniques, e.g., in a high salt elution buffer and then dialyzed into a lower salt buffer for use or by changing pH or other components depending on the affinity matrix utilized, or can be competitively removed using the naturally occurring substrate of the affinity moiety.

The desired degree of final purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Optionally, the polypeptide of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

The invention also optionally encompasses further formulating the polypeptides. By the term “formulating” is meant that the polypeptides can be buffer exchanged, sterilized, bulk-packaged, and/or packaged for a final user. For purposes of the invention, the term “sterile bulk form” means that a formulation is free, or essentially free, of microbial contamination (to such an extent as is acceptable for food and/or drug purposes), and is of defined composition and concentration. The term “sterile unit dose form” means a form that is appropriate for the customer and/or patient administration or consumption. Such compositions can comprise an effective amount of the polypeptide, in combination with other components such as a physiologically acceptable diluent, carrier, or excipient. The term “physiologically acceptable” means a non-toxic material that does not interfere with the effectiveness of the biological activity of the active ingredient(s).

Formulations suitable for administration include aqueous and non-aqueous sterile injection solutions which may contain anti-oxidants, buffers, bacteriostats, and solutes which render the formulation isotonic with the blood of the recipient; and aqueous and non-aqueous sterile suspensions which may include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants, and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871, 4,501,728, 4,837,028, and 4,737,323. Such compositions will influence the physical state, solubility, stability, rate of in vivo release, and rate of in vivo clearance, and are thus chosen according to the intended application, so that the characteristics of the carrier will depend on the selected route of administration. Sustained-release forms suitable for use include, but are not limited to, polypeptides that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

The invention having been described, the following examples are offered by way of illustration, and not limitation.

### EXAMPLE 1

### Results Using 0.5 mM Hydrocinnamic Acid

CHO cells were transfected with an expression vector that places the gene encoding the fluorescent reporter gene DsRed (BD Biosciences Clontech, Palo Alto, Calif.) under the control of an EASE/CMV promoter system as described in U.S. Pat. No. 6,027,915, incorporated by reference herein. Pools of cells were selected in -GHT medium for presence of the expression vector, and then plated in a 96 well format in serum-free medium. Hydrocinnamic acid was added to test wells at 0.5 millimolar, the temperature decreased from 37° C. to 31° C., and culture continued for 6 days at the reduce temperature. DsRed fluorescence was assayed on a Wallac Victor2 multilabel microplate reader (PerkinElmer Life Sciences, Boston, Mass.). Over a total of 3 experiments, there was an average 60% increase in DsRed expression per viable cell relative to control.

The induction capacity of this compound was then tested in a different format on a different recombinant protein. In this case, the cells were a CHO cell line that expresses a soluble form of the IL1-receptor type II (see U.S. Pat. No. 6,521,740, incorporated by reference herein). Cells were grown in serum-free medium in shake flasks, hydrocinnamic acid was added to a concentration of 0.5 millimolar, and incubation continued at 31° C. for 9 to 10 days. Over a total of 5 experiments, the average increase in IL1RII expression relative to control was 22%.

Another cell line tested was a CHO cell that expresses a soluble form of a TNF receptor, TNFR:Fc (U.S. Pat. No. 5,605,690, incorporated by reference herein). After growing the cells at 37° C. in serum-containing medium, the cells were switched to shake flasks containing serum-free medium and 0.5 mM Hydrocinnamic acid and incubated for a further 7 days under these induction conditions at a reduced temperature. At the end of the incubation period, the cells grown in medium containing hydrocinnamic acid showed a 20% increase in TNFR:Fc expression relative to control cells.

In addition, CHO cell pools transfected with an expression vector encoding an antibody against the IL4 receptor were tested (see U.S. Pat. No. 5,717,072, incorporated by reference herein). Unamplified pools were plated in serum-free medium in 96 well plates and incubated with inducer for 4 days at 37° C. Over a total of 3 different experiments, the pools exposed to hydrocinnamic acid exhibited an average 5% increase in antibody expression relative to control.

### EXAMPLE 2

### Results Using 0.5 mM 3-(4-methylphenyl)propionic acid

The pools of CHO cells were transfected with an expression vector that places the gene encoding the fluorescent reporter gene DsRed described above in Example 1 were also tested for induction by 0.5 mM 3-(4-methylphenyl)propionic acid in a 96 well format. Pools were incubated for 6 days at 31° C. Over at least 3 different experiments, the pools incubated with 3-(4-methylphenyl)propionic acid averaged a 70% increase in DsRed expression per viable cell relative to control.

The induction capacity of this compound was then confirmed in a shake flask format using the CHO cell line that expresses a soluble form of the IL1-receptor type II. Cells were grown in serum-free medium in shake flasks, 3-(4-methylphenyl)propionic acid was added to a concentration of 0.5 millimolar, and incubation continued at 31° C. for 9 to 10 days. Over a total of 2 experiments, the average increase in IL1RII expression relative to control was 15%.

### EXAMPLE 3

### Results Using 0.5 mM 4-phenylbutyric acid

The pools of CHO cells were transfected with an expression vector that places the gene encoding the fluorescent reporter gene DsRed described above in Example 1 were also tested for induction by 0.5 mM 4-phenylbutyric acid in a 96 well format. Pools were incubated for 6 days at 31° C. Over 3 different experiments, the pools incubated with 4-phenylbutyric acid averaged a 60% increase in DsRed expression per viable cell relative to control.

The induction capacity of this compound was then confirmed in a shake flask format using the CHO cell line that expresses a soluble form of the IL1-receptor type II. Cells were grown in serum-free medium in shake flasks, 4-phenylbutyric acid was added to a concentration of 0.5 millimolar, and incubation continued at 31° C. for 9 to 10 days. The cell line grown in the presence of 4-phenylbutyric acid increased IL1RII expression relative to control by 21%.

### EXAMPLE 4

### Results Using 0.5 mM 4-(4-aminophenyl)butyric acid

The pools of CHO cells were transfected with an expression vector that places the gene encoding the fluorescent reporter gene DsRed described above in Example 1 were also tested for induction by 0.5 mM 4-(4-aminophenyl)butyric acid in a 96 well format. Pools were incubated for 6 days at 31° C. Over 3 different experiments, the pools incubated with 4-(4-aminophenyl)butyric acid averaged a 70% increase in DsRed expression per viable cell relative to control.

The induction capacity of this compound was then confirmed in a shake flask format using the CHO cell line that expresses a soluble form of the IL1-receptor type II. Cells were grown in serum-free medium in shake flasks, 4-(4-aminophenyl)butyric acid was added to a concentration of 0.5 millimolar, and incubation continued at 31° C. for 9 to 10 days. Over 3 experiments, the cell line grown in the presence of 4-(4-aminophenyl)butyric acid increased IL1RII expression relative to control by an average of 22%.

### EXAMPLE 5

### Results Using 0.5 mM 5-phenylvaleric acid

The pools of CHO cells were transfected with an expression vector that places the gene encoding the fluorescent reporter gene DsRed described above in Example 1 were also tested for induction by 0.5 mM 5-phenylvaleric acid in a 96 well format. Pools were incubated for 6 days at 31° C. Over 3 different experiments, the pools incubated with 5-phenylvaleric acid averaged a 40% increase in DsRed expression per viable cell relative to control.

The induction capacity of this compound was then confirmed in a shake flask format using the CHO cell line that expresses a soluble form of the IL1-receptor type II. Cells were grown in serum-free medium in shake flasks, 5-phenylvaleric acid was added to a concentration of 0.5 millimolar, and incubation continued at 31° C. for 9 to 10 days. The cell line grown in the presence of 5-phenylvaleric acid increased IL1RII expression relative to control by 44%.

Another cell line tested was the CHO cell that expresses TNFR:Fc (etanercept) described above in Example 1. After growing the cells at 37° C. in serum-containing medium, the cells were switched to shake flasks containing serum-free medium and 0.5 millimolar 5-phenylvaleric acid and incubated for a further 7 days under these induction conditions at a reduced temperature. At the end of the incubation period, the cells grown in medium containing 5-phenylvaleric acid showed a 33% increase in TNFR:Fc expression relative to control cells.

In addition, CHO cell pools transfected with an expression vector encoding an antibody against the IL4 receptor were tested. Unamplified pools were plated in a 96 well plate and incubated with inducer for 4 days at 37° C. Over a total of 2 different experiments, the pools exposed to 5-phenylvaleric acid exhibited an average 40% increase in antibody expression relative to control. Most of this increase occurred on the final day of culture as there was no increase on day 3.

### EXAMPLE 6

### 1.0 mM N-butylacetamide

The pools of CHO cells transfected with an expression vector that places the gene encoding the fluorescent reporter gene DsRed described above in Example 1 were also tested for induction by 1.0 millimolar N-butylacetamide in a 96 well format. Pools were incubated for 6 days at 31° C. Over 2 different experiments, the pools incubated with N-butylacetamide acid averaged a 77% increase in DsRed expression per viable cell relative to control.

### EXAMPLE 7

### 10/20 μM Hexanohydroxamic acid (HHA)

The pools of CHO cells transfected with an expression vector that places the gene encoding the fluorescent reporter gene DsRed described above in Example 1 were also tested for induction by 10 micromolar hexanohydroxamic acid (HHA) in a 96 well format. Pools were incubated for 6 days at 31° C. The pools incubated with HHA exhibited a 48% increase in DsRed expression relative to the control.

The induction capacity of this compound was then confirmed in a shake flask format using the CHO cell line that expresses a soluble form of the IL1-receptor type II. Cells were grown in serum-free medium in shake flasks, HHA was added to a concentration of 10 micromolar, and incubation continued at 31° C. for 9 days. The cell line grown in the presence of 10 micromolar HHA increased IL1RII expression relative to control by 19%.

Another cell line tested was the CHO cell that expresses TNFR:Fc (etanercept) described above in Example 1. After growing the cells at 37° C. in serum-containing medium, the cells were switched to shake flasks containing serum-free medium and HHA added at either 10 micromolar or 20 micromolar, and incubated for a further 7 days under these induction conditions at a reduced temperature. At the end of the incubation period, the cells grown in medium containing 10 micromolar HHA showed a 19% increase in TNFR:Fc expression relative to control cells, while those grown in 20 micromolar HHA showed an 11% increase in TNFR:Fc expression relative to control cells.

In addition, CHO cell pools transfected with an expression vector encoding an antibody against the IL4 receptor were tested. Unamplified pools in serum-free medium were plated in a 96 well plate and incubated with inducer for 4 days at 37° C. Over 2 different experiments, the pools exposed to HHA did not have a significant increase in antibody expression relative to control.

### EXAMPLE 8

### 10 μM HHA Plus 1 mM HMBA

In these experiments, a combination of two different inducers was used. As can be seen from the results below, combining inducers from different classes can result in greater than additive increases on induction.

The pools of CHO cells transfected with an expression vector that places the gene encoding the fluorescent reporter gene DsRed described above in Example 1 were also tested for induction by 10 micromolar HHA and 1 millimolar HMBA in a 96 well format. Pools were incubated for 6 days at 31° C. The pools incubated with just HMBA exhibited a 20% increase, while the pools incubated with the combination of HHA and HMBA exhibited a 98% increase in DsRed expression relative to the control. As noted in Example 7, HHA alone only resulted in a 48% increase. Thus, these two compounds have at least an additive effect when used in combination.

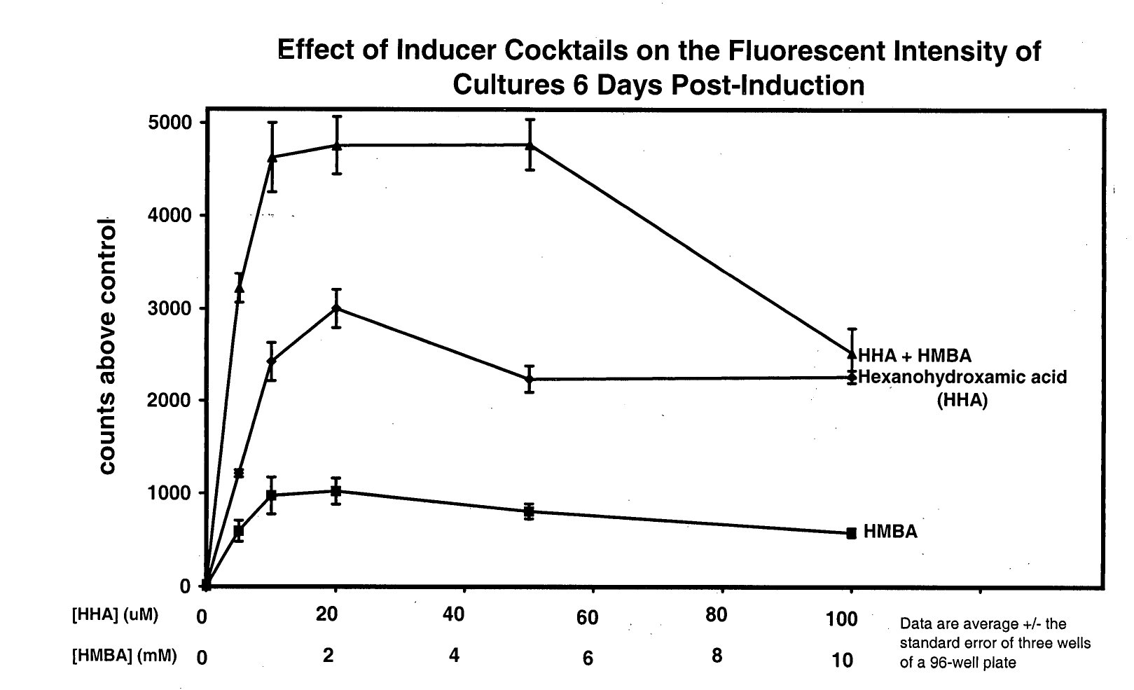

Data from a similar experiment in which the cells were incubated at various concentrations of HHA and HMBA are illustrated in FIG. 1. As can be seen from this graph, concentrations of HHA from about 5 micromolar to about 100 micromolar, and concentrations of HMBA from about 0.5 millimolar to about 10 millimolar when used indivually could increase expression of the fluorescent marker DsRed. When these compounds were combined, however, expression was increased significantly more.

Another cell line tested was the CHO cell that expresses TNFR:Fc (etanercept) described above in Example 1. After growing the cells at 37° C. in serum-containing medium, the cells were switched to shake flasks containing serum-free medium. HHA was added at 10 micromolar and HMBA was added at 1 millimolar, and the cells incubated for a further 7 days under these induction conditions at a reduced temperature. At the end of the incubation period, the cells grown in medium containing 1.0 millimolar HMBA showed a 14% increase in TNFR:Fc expression relative to control cells, while those grown in the combination of 10 micromolar HHA plus 1.0 millimolar HMBA showed a 26% increase in TNFR:Fc expression relative to control cells.

In addition, CHO cell pools transfected with an expression vector encoding an antibody against the IL4 receptor were tested. Unamplified pools were plated in serum-free medium in 96 well plates and incubated with 10 micromolar HHA plus 1.0 millimolar HMBA for 4 days at 37° C. Over 2 different experiments, the pools exposed to the combination of 10 micromolar HHA plus 1.0 millimolar HMBA showed a 32% increase in antibody expression relative to control. However, when either compound was used individually, there was not a significant increase in antibody expression. This result suggests that these compounds can act synergistically to increase recombinant protein expression.

### EXAMPLE 9

### 10 μM 3-phenylpropionohydroxamic acid Plus 1 mM HMBA

CHO cell pools transfected with an expression vector encoding an antibody against the IL4 receptor were tested with another combination of compounds, in this case, 10 micromolar 3-phenylpropionohydroxamic acid plus 1 millimolar HMBA. Unamplified pools were plated in serum-free medium in 96 well plates and incubated with this combination for 4 days at 37° C. The pools exposed to the combination of 3-phenylpropionohydroxamic acid plus 1 millimolar HMBA showed a 40% increase in antibody expression relative to control. However, when either compound was used individually, there was not a significant increase in antibody expression. This result, especially when taken in combination with the result from Example 8, suggests that a combination of an acetamide and a hydroxamic acid compound can act synergistically to increase recombinant protein expression.

The foregoing description of specific embodiments reveals the general nature of the invention so that others can readily modify and/or adapt such embodiments for various applications without departing from the generic concepts presented herein. Any such adaptions or modifications are intended to be embraced within the meaning and range of equivalents of the disclosed embodiments. Phraseology and terminology employed herein are for the purpose of description and not of limitation.

What is claimed is:

1. A method comprising:
culturing a mammalian cell in a culture medium containing an aromatic carboxylic acid compound, wherein the mammalian cell secretes a polypeptide of interest and wherein the presence of the aromatic carboxylic acid compound increases production of the polypeptide of interest; and
separating the polypeptide of interest from the mammalian cell.

2. The method of claim 1, further comprising lowering the temperature of the culture medium to a temperature of less than 37° C.

3. The method of claim 2, wherein the temperature is lowered to about 29° C. to about 34° C.

4. The method of claim 1, wherein the mammalian cell expresses the polypeptide of interest under the control of a CMV promoter.

5. The method of claim 1, wherein the aromatic carboxylic acid compound is selected from the group consisting of hydrocinnamic acid, 3-(4-methylphenyl)propionic acid, 4-phenylbutyric acid, 4-(4-aminophenyl)butyric acid, 3-(4-aminophenyl)propionic acid; 3-(4-fluorophenyl)propionic acid; 2-thienylacetic acid, and 5-phenylvaleric acid.

6. The method of claim 1, wherein the polypeptide is a recombinant fusion polypeptide.

7. The method of claim 1, wherein the polypeptide is a human or humanized antibody.

8. The method of claim 1, wherein the concentration of the aromatic carboxylic acid compound in the culture is from about 0.001 millimolar to about 3 millimolar.

9. The method of claim 1, further comprising adding an acetamide compound to the culture.

10. The method of claim 9, wherein the acetamide compound is hexamethylenebisacetamide (HMBA) and/or N-butylacetamide.

11. The method of claim 1, further comprising adding a hydroxamic acid compound to the culture.

12. The method of claim 11, wherein the hydroxamic acid compound is hexanohydroxamic acid (HHA), benzohydroxamic acid, octane-1,8-dihydroxamic acid and/or 3-phenylpropionohydroxamic acid.

13. The method of claim 1, wherein the mammalian cell is a CHO cell.

14. The method of claim 13, wherein the CHO cell is exposed to the aromatic carboxylic acid compound for at least about 5 days.

15. The method of claim 1, wherein the culture medium is serum free.

16. The method of claim 1, further comprising purifying the polypeptide.

17. The method of claim 1,
wherein the mammalian cell is cultured in a growth phase at a first temperature from about 35° C. to about 38° C. before it is shifted to a production phase at a second temperature from about 29° C. to about 36° C. and
wherein the aromatic carboxylic acid compound is added after the shift to the production phase.

18. A method comprising:
culturing a mammalian cell in a culture medium containing a non-hybrid polar acetamide compound, wherein the mammalian cell secretes a polypeptide of interest and wherein the presence of the acetamide compound increases production of the polypeptide of interest; and
separating the polypeptide of interest from the mammalian cell.

19. The method of claim 18, further comprising lowering the temperature of the culture medium to a temperature of less than 37° C.

20. The method of claim 19, wherein the temperature is lowered to about 29° C. to about 34° C.

21. The method of claim 18, wherein the mammalian cell expresses the polypeptide of interest under the control of a CMV promoter.

22. The method of claim 18, wherein the acetamide compound is N-butylacetamide.

23. The method of claim 18, wherein the polypeptide is a recombinant fusion polypeptide.

24. The method of claim 18, wherein the polypeptide is a human or humanized antibody.

25. The method of claim 18, wherein the concentration of the acetamide compound in the culture is from about 0.001 millimolar to about 3 millimolar.

26. The method of claim 18, further comprising adding a hydroxamic acid compound to the culture.

27. The method of claim 26, wherein the hydroxamic acid compound is hexanohydroxamic acid (HHA) and/or 3-phenylpropionohydroxamic acid.

28. The method of claim 18, wherein the mammalian cell is a CHO cell.

29. The method of claim 28, wherein the CHO cell is exposed to the non-hybrid polar acetamide compound for at least about 5 days.

30. The method of claim 18, wherein the culture medium is serum free.

31. The method of claim 18, further comprising purifying the polypeptide.

32. The method of claim 18,
wherein the mammalian cell is cultured in a growth phase at a first temperature from about 35° C. to about 38° C. before it is shifted to a production phase at a second temperature from about 29° C. to about 36° C. and
wherein the non-hybrid polar acetamide compound is added after the shift to the production phase.

33. A method comprising:
culturing a mammalian cell in a culture medium containing a hydroxamic acid compound, wherein the mammalian cell secretes a polypeptide of interest and wherein the presence of the hydroxamic acid compound increases production of the polypeptide of interest; and
separating the polypeptide of interest from the mammalian cell.

34. The method of claim 33, further comprising lowering the temperature of the culture medium to a temperature of less than 37° C.

35. The method of claim 34, wherein the temperature is lowered to about 29° C. to about 34° C.

36. The method of claim 33, wherein the mammalian cell expresses the polypeptide of interest under the control of a CMV promoter.

37. The method of claim 33, wherein the hydroxamic acid compound is hexanohydroxamic acid (HHA) and/or 3-phenylpropionohydroxamic acid.

38. The method of claim 33, wherein the polypeptide is a recombinant fusion polypeptide.

39. The method of claim 33, wherein the polypeptide is a human or humanized antibody.

40. The method of claim 33, wherein the concentration of the hydroxamic acid compound in the culture is from about 0.001 millimolar to about 3 millimolar.

41. The method of claim 33, further comprising adding an acetamide compound to the culture.

42. The method of claim 41, wherein the acetamide compound is hexamethylenebisacetamide (HMBA) and/or N-butylacetamide.

43. The method of claim 33, wherein the mammalian cell is a CHO cell.

44. The method of claim 43, wherein the CHO cell is exposed to the hydroxamic acid compound for at least about 5 days.

45. The method of claim 33, wherein the culture medium is serum free.

46. The method of claim 33, further comprising purifying the polypeptide.

47. The method of claim 33,
wherein the mammalian cell is cultured in a growth phase at a first temperature from about 35° C. to about 38° C. before it is shifted to a production phase at a second temperature from about 29° C. to about 36° C. and
wherein the hydroxamic acid compound is added after the shift to the production phase.

48. A method for producing a recombinant polypeptide comprising:
culturing a CHO cell that has been genetically engineered to produce the recombinant polypeptide; and
adding to the culture medium at least one compound selected from the group consisting of an aromatic carboxylic acid, a non-hybrid polar acetamide, and a hydroxamic acid,
wherein the addition of the compound increases the production of the recombinant polypeptide.

49. The method of claim 48,
wherein the CHO cell is the progeny of a cell has been transformed with a recombinant vector encoding the recombinant polypeptide and
wherein the recombinant vector comprises a CMV promoter.

50. The method of claim 48, wherein the compound is added to the culture medium at a concentration of from about 0.001 millimolar to about 3 millimolar.

51. The method of claim 48, further comprising collecting the recombinant polypeptide from the medium.

52. The method of claim 51, further comprising formulating the recombinant polypeptide.

53. The method of claim 51, further comprising multiple additions of the compound.

54. The method of claim 48, wherein the CHO cell is cultured at a temperature from about 29° C. to about 35° C.

55. The method of claim 54,
wherein the CHO cell is cultured at a first temperature from about 36° C. to about 38° C. before it is shifted to a second temperature from about 29° C. to about 35° C. and
wherein the compound is added after the shift from the first temperature to the second temperature.

56. A culture comprising a CHO cell genetically engineered to produce a polypeptide, a production medium, and at least one compound selected from the group consisting of an aromatic carboxylic acid, a non-hybrid polar acetamide, and a hydroxamic acid.

57. The culture of claim 56, wherein the concentration of the compound is from about 0.01 millimolar to about 3 millimolar.

58. The culture of claim 56, wherein the production medium is serum-free.
